(19) United States
(12) Patent Application Publication
(10) Pub. No.: US 2023/0181971 A1
(43) Pub. Date: Jun. 15, 2023

(54) SYSTEM AND METHOD FOR ANALYZING ONE OR MOVEMENTS OF A MOVEABLE ENTITY

(71) Applicant: TrackIT Athletes Inc., Brighton, MA (US)

(72) Inventors: Jaime Stephen, Brighton, MA (US); Taylor Stephen, Houston, TX (US); Anuar Jauregui, The Woodlands, TX (US); Max Luu, Beaverton, OR (US); Martin Orellana, Spring, TX (US); Rishab Narendra, Brighton, MA (US); Samuel Sierra, Brighton, MA (US)

(73) Assignee: TrackIT Athletes Inc., Brighton, MA (US)

(21) Appl. No.: 18/065,173

(22) Filed: Dec. 13, 2022

Related U.S. Application Data
(60) Provisional application No. 63/289,381, filed on Dec. 14, 2021.

(51) Int. Cl. A63B 24/00; A63B 71/06

(52) U.S. Cl. CPC A63B 24/0006; A63B 71/0622; A63B 2024/0015; A63B 2220/803; A63B 2220/806

(57) ABSTRACT

A system and method for analyzing one or more movements of moveable entities (i.e., persons or animals) is disclosed. The system includes a processor that is in communication with a camera. The processor is configured to determine one or more skeletal locations of the one or more moveable entities performing a movement based on a plurality of images captured by the camera. The processor is further configured to determine one or more kinematic measurements of the one or more skeletal locations of the one or more moveable entities. The processor is further configured to compare the one or more kinematic measurements of the one or more moveable entities with an ideal kinematic movement for the one or more moveable entities. In addition, the processor is configured to provide an analysis based on the comparison.

### CROSS-REFERENCE TO RELATED APPLICATIONS

This application claims priority to U.S. Pat. Application Number US 63/289,381 filed on Dec. 14, 2021, the complete disclosure of which, in its entirely, is herein incorporated by reference.

### FIELD

This disclosure relates to the field of movement monitoring. More specifically, the field relates to a system and method for analyzing one or more movements of a moveable entity and an object associated with the moveable entity while performing one or more movements and providing one or more suggestions for improvement. Examples of the movements may be a person playing sports, performing athletic activities, or other motion-related actions.

### BACKGROUND

People who practice sports or perform movement-related activities such as dancing, performing maintenance or construction, physical therapy, and the like often take time and effort to reach an appropriate or high level. To achieve such levels, people generally engage with mentors/coaches/trainers who assist them in teaching the correct forms and techniques while playing the respective sport(s) or performing the movements. The trainer monitors and corrects the errors or faults that occur while the person is performing the movement and instructs the correct form/manner for performing the respective movement. Thus, people depend upon the trainer’s skill, experience, and patience while practicing.

However, the act of depending on trainers may be less than ideal. A single trainer may train/coach multiple persons simultaneously, due to which the person may get personal coaching in fewer instances. Further, when people perform complex and fast movements, it may be challenging for the trainer to analyze multiple movements accurately. This may cause them to miss details that would enhance training instructions for the person.

### SUMMARY

The present disclosure discloses a system for analyzing one or more movements of a person. The system includes a processor in communication with a camera. The processor is configured to determine one or more skeletal locations of the person performing a movement based on a plurality of images captured by the camera. The processor is further configured to determine one or more kinematic measurements of the one or more skeletal locations of the person. The processor is further configured to compare the one or more kinematic measurements of the person with an ideal kinematic movement for the person. In addition, the processor is configured to provide an analysis of the person based on the comparison.

In accordance with the aspects of the disclosure, the ideal kinematic movement is based on at least one of a size, a weight, an age, a gender, a limb diameter, a limb density, a body diameter, a body density, and a skeletal makeup of the person.

In accordance with the aspects of the disclosure, the processor is further configured to determine one or more location points of an object associated with the person. The processor is further configured to determine one or more kinematic measurements of the object based on the one or more location points.

In accordance with the aspects of the disclosure, the processor is further configured to determine the first location of each skeletal location of the person. The processor is further configured to determine one or more subsequent locations of each skeletal location while the person moves at intervals. The processor is further configured to receive the plurality of images of the person captured by the camera at each interval. In addition, the processor is configured to determine one or more kinematic measurements at the first location and subsequent locations for a selected image at each interval.

In accordance with the aspects of the disclosure, the system further comprises a display that displays the one or more kinematic measurements while the person is performing the movement.

In accordance with the aspects of the disclosure, the one or more kinematic measurements are further determined based on one or more sensors that track movement of the camera.

In accordance with the aspects of the disclosure, the processor is further configured to determine a first object location of the object that is associated with the person. The processor is further configured to determine one or more subsequent locations of the object at intervals. The processor is further configured to receive the plurality of images of the object captured by the camera at each interval. In addition, the processor is configured to determine one or more kinematic measurements at the first object location and subsequent object locations for a selected image at each interval.

In accordance with the aspects of the disclosure, the system further comprises a display that displays the one or more kinematic measurements of the object while the person is performing the movement.

In accordance with the aspects of the disclosure, the system further comprises one or more sensors that measure motion of the moveable entity. The one or more kinematic measurements are further based on the one or more sensors.

In accordance with the aspect of the disclosure, the processor is further configured to receive an input from the person based on the analysis. The input comprises at least one of a voice message, verbal message, email message, button, Bluetooth, QR code, gesture signal, or a combination thereof.

In accordance with the aspects of the disclosure, the processor is further configured to provide a trend based on an analysis of two or more movements of the person.

In accordance with the aspects of the disclosure, the trend comprises a rating based on the one or more determined kinematic movements.

The present disclosure also discloses a method for analyzing one or more movements of a person. The steps of the method may be executed by a processor in communication with a camera. The method includes determining one or more skeletal locations of the person performing a movement based on a plurality of images captured by the camera. The method further includes determining one or more kinematic measurements of the one or more skeletal locations of the person. The method further includes comparing the one or more kinematic measurements of the person with an ideal kinematic movement for the person. In addition, the method includes providing an analysis of the person based on the comparison.

In accordance with the aspects of the disclosure, the ideal kinematic movement is based on at least one of a size, a weight, an age, a gender, a limb diameter, a limb density, a body diameter, a body density, and a skeletal makeup of the person.

In accordance with the aspects of the disclosure, the method further comprises determining one or more location points of an object that is associated with the person while performing the movement and determining one or more kinematic measurements of the object based on the one or more location points.

In accordance with the aspects of the disclosure, the method further comprises determining a first location of each skeletal location of the person and determining one or more subsequent location of each skeletal location while the person moves at intervals. The method further comprises receiving the plurality of images of the person captured by the camera at each interval. In addition, the method further comprises determining one or more kinematic measurements at the first location and subsequent locations for a selected image at each interval.

In accordance with the aspects of the disclosure, the method further comprises displaying the one or more kinematic measurements at least while the person is performing the movement or after the person has performed the movement.

In accordance with the aspects of the disclosure, the method further comprises determining a first object location of the object that is associated with the person and determining one or more subsequent locations of the object at intervals. The method further comprises receiving the plurality of images of the object captured by the camera at each interval. In addition, the method comprises determining one or more kinematic measurements at the first object location and subsequent object locations for a selected image at each interval.

In accordance with the aspects of the disclosure, the method further comprises displaying the one or more kinematic measurements of the object at least while the person is performing the movement or after the person has performed the movement.

The present disclosure also discloses one or more non-transitory computer-readable storage mediums storing one or more sequences of instructions. The one or more sequence of instructions is executed by a processor configured to determine one or more skeletal locations of a person performing a movement based on a plurality of images captured by a camera in communication with the one or more processors. The one or more processors are further configured to determine one or more kinematic measurements of the one or more skeletal locations of the person. The one or more processors are further configured to determine one or more location points of an object associated with the person while performing the movement. The one or more processors are further configured to determine one or more kinematic measurements of the object based on the one or more location points. The one or more processors are further configured to compare the one or more kinematic measurements of the person and the object with an ideal kinematic movement. In addition, the one or more processors are configured to provide an analysis of the person based on the comparison.

### BRIEF DESCRIPTION OF THE DRAWINGS

FIG. 1 illustrates a block diagram of the components of the system according to an embodiment herein;

FIG. 2 illustrates an exploded view of the processor of FIG. 1 according to an embodiment herein;

FIG. 3 illustrates a sign-in screen in which the person logs in to a movement analyzing application installed in their respective device by providing their credentials according to an embodiment herein;

FIG. 4 illustrates a sign-up screen in which the person creates an account in the movement analyzing application according to an embodiment herein;

FIG. 5 illustrates a main screen of the movement analyzing application upon login according to an embodiment therein;

FIG. 6 illustrates a log screen of the movement analyzing application upon clicking the log option of the main screen in FIG. 5 according to an embodiment herein;

FIG. 7 illustrates a group screen of the movement analyzing application upon clicking the groups option of the main screen in FIG. 5 according to an embodiment herein;

FIG. 8 illustrates a performance screen of the movement analyzing application upon clicking the performance option of the main screen in FIG. 5 according to an embodiment herein;

FIG. 9 illustrates a view of the person in which a plurality of markers are secured to their body parts according to an embodiment herein;

FIG. 10A illustrates views of the person in which one or more skeletal locations are shown according to an embodiment herein;

FIG. 10B illustrates a left-side view of the one or more skeletal locations shown in FIG. 10A as applied to a human individual;

FIG. 10C illustrates a right-side view of the one or more skeletal locations shown in FIG. 10A as applied to a human individual;

FIGS. 11A-E illustrate sequences of the person captured by the camera while performing a weightlifting exercise according to an embodiment herein;

FIG. 12 illustrates an analysis view of the person illustrating kinematic measurements determined for movements performed by the person while weightlifting according to an embodiment herein;

FIG. 13 illustrates an analysis view of the person illustrating kinematic measurements determined for movements performed by the person while playing discus according to an embodiment herein;

FIG. 14 illustrates an analysis view of the person illustrating kinematic measurements determined for movements performed by the person while playing volleyball according to an embodiment herein;

FIG. 15 illustrates an analysis view of the person illustrating kinematic measurements determined for movements performed by the person while playing tennis according to an embodiment herein;

FIG. 16 illustrates a flowchart illustrating a method for analyzing one or more movements of a person according to an embodiment herein;

FIG. 17 illustrates a flowchart illustrating a method for displaying kinematic measurements of one or more skeletal locations of the person while performing the movement at intervals according to an embodiment herein;

FIG. 18 illustrates a flowchart illustrating a method for displaying kinematic measurements of one or more locations of the object associated with the person while performing the movement according to an embodiment herein; and

FIG. 19 illustrates a schematic of an embodiment of a computer system that may be implemented to carry out the disclosed subject matter.

### DETAILED DESCRIPTION

Embodiments, of the present disclosure, will now be described with reference to the accompanying drawing.

Embodiments are provided so as to convey the scope of the present disclosure thoroughly and fully to the person skilled in the art. Numerous details, are set forth, relating to specific components, and methods, to provide a complete understanding of embodiments of the present disclosure. It will be apparent to the person skilled in the art that the details provided in the embodiments may not be construed to limit the scope of the present disclosure. In some embodiments, well-known processes, well-known apparatus structures, and well-known techniques are not described in detail.

The terminology used, in the present disclosure, is for the purpose of explaining a particular embodiment and such terminology may not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, elements, modules, units and/or components, but do not forbid the presence or addition of one or more other features, elements, components, and/or groups thereof. The particular order of steps disclosed in the method and process of the present disclosure is not to be construed as requiring their performance as described or illustrated. It is also to be understood that additional or alternative steps may be employed.

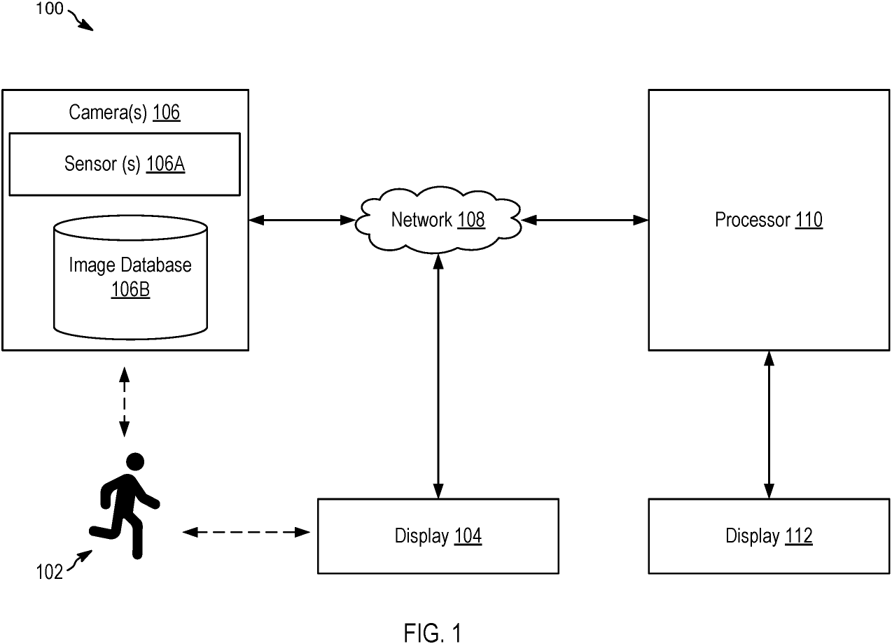

FIG. 1 illustrates a block diagram of the components of the system 100 for analyzing one or more movements of a moveable entity according to an embodiment herein. As used herein, the term “moveable entity” refers to any object or organism that exhibits independent movement of a body, limbs, stalk, trunk, branches, appendages, or the like. In an example, the moveable entity is a moveable entity. The moveable entity may include people, non-human organisms, robots, mechanical devices, any living thing that has the ability to move, and any non-living object that exhibits movement. Examples of non-human organisms include but are not limited to dogs, cats, elephants, pigs, cows, rats, whales, doves, pigeons, parrots, falcons, eagles, turkeys, fish, squid, octopi, spiders, lobsters, and crabs. In various embodiments, the moveable entity may comprise more than one entity. For example, the disclosed subject matter may provide a movement analysis of a dance movement by two or more dancers. The one or more movements may be movements performed while playing sports, performing athletic activities, or other motion-related actions. In an example, the sports may include squash, racquetball, tennis, hockey, cricket, badminton, bowling, golf, football, baseball, table tennis, jai alai, and the like. The athletic activities may include squats, bench press, shotput, discus, javelin, and the like. The other motion-related activities may include weightlifting, dancing, jumping, running, skating, skiing, snowboarding, physical therapy, and the like.

As shown, the system 100 includes a device 104 associated with the person 102, a camera(s) 106, a network 108, a processor 110, and a display 112. The person 102 may be a professional athlete, coach, school student, college student, gym trainer, gym member, aged person, or any person interested in getting their sports technique or movements analyzed. In an example, the device 104 may be a personal computer (PC), mobile device, smartphone, tablet, personal device assistant (PDA), and the like. In an example, the camera(s) 106 may be a personal computer (PC), mobile device, smartphone, tablet, personal device assistant (PDA), an RGB (red, green, blue) camera, a stereo camera that is capable of capturing 3D pictures, a depth camera, and the like. Further, the display 112 may be a cathode-ray tube (CRT), a liquid-crystal display (LCD), a LED display, plasma, OLED, a cell phone/mobile phone display, and the like.

In an embodiment, the camera(s) 106 is configured to capture a plurality of images of the person 102 while they are performing their movements. More than one camera may be used for capturing the images of the person 102 while they are performing their movement. The camera(s) 106 includes a sensor(s) 106A and an image database 106B. In an example, the sensor(s) 106A may include a visible imaging sensor, radar, LIDAR, UV, accelerometer, GPS, and the like. The functionality and use of the sensor(s) 106A will be explained in further detail below. The image database 106B stores the plurality of images captured by the camera(s) 106.

The camera(s) 106 encompasses an action area that the person 102 is interested in monitoring and tracking. Depending on the movement being performed by the person 102, the camera(s) 106 is generally placed in locations where minimal shadow effects of the person 102 are present. The camera(s) 106 may be placed on a stand that is in front of the person 102, behind the person 102, or at the sides of the person 102. Also, the camera(s) 106 is generally placed at a height between a shoulder level and waist level of the person 102. This is to ensure that the images captured by the camera(s) 106 are of good quality. Further, the camera(s) 106 is in communication with the processor 110 via the network 108. In an example, the camera(s) 106 and processor 110 may be connected or paired with each other via Bluetooth. Also, the processor 110 and display 112 may be connected or paired with each other via Bluetooth.

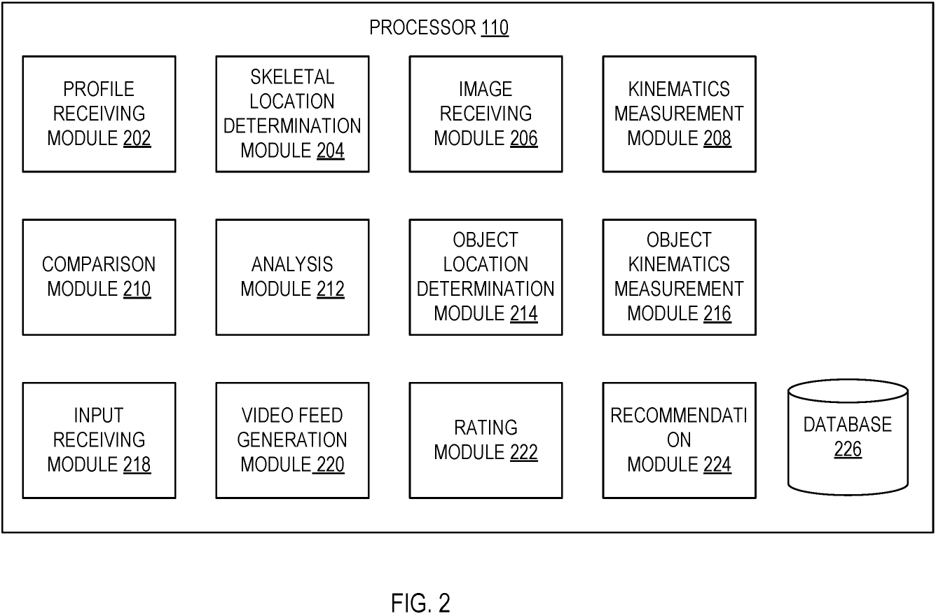

FIG. 2 illustrates an exploded view of the processor 110 of FIG. 1 according to an embodiment herein. As shown, the processor 110 includes a profile receiving module 202, a skeletal location determination module 204, an image receiving module 206, a kinematics measurement module 208, a comparison module 210, an analysis module 212, an object location determination module 214, an object kinematics measurement module 216, an input receiving module 218, a video feed generation module 220, a rating module 222, a recommendation module 224, and a database 226. The functionality of each module is explained in further detail below.

In an embodiment, the profile receiving module 202 receives a profile of the person 102 at the time of registration with the application (movement analyzing application) installed in their respective device 104. The profile of each person 102 is stored in the database 226. Also, the person 102 may update their profile details at any point in time, and these changes are instantly updated in the database 226. In an example, the profile of each person 102 may include information such as their name, photo, contact details (phone number, email ID), age, gender, weight, address, sports/activities that they are interested in playing/performing, awards/recognitions achieved in the past for such sports or activities, future athletic goals, areas of improvement, the confidence level for each sport/activity played/performed by them, and the like.

In an embodiment, the skeletal location determination module 204 determines one or more skeletal locations of the person 102 based on the plurality of images captured by the camera(s) 106. In an exemplary embodiment, the term “skeletal locations” refers to a simulated armature with bones and joints that may be used by computer software to describe the movement of the person or non-human moveable entity. The skeletal locations do not need to correspond to actual skeletal bones on the moveable entity. In various embodiments, the skeletal locations may be determined for moveable entities that do not have bones such as octopi or robots. In an example for a person, the one or more skeletal locations may include at least one of ankle positions, knee positions, hip positions, wrist positions, elbow positions, shoulder positions, eye positions, and the like of the person 102. The skeletal location determination module 204 obtains the one or more skeletal locations of the person 102 at a first location and then at subsequent locations. The first location may correspond to the position of the person 102 before they start moving or at the start of their movement/exercise/activity. The one or more subsequent locations correspond to the position of the person 102 once they start performing their movement. For example, if the person 102 is performing a bar lifting movement exercise, the first location may correspond to the position/location when the bar is on the ground, and the person 102 has gripped both hands on the respective sides of the bar. The one or more subsequent locations may correspond to positions/locations of the person 102 as he/she lifts the bar from the ground and places the bar above his/her shoulders.

The skeletal location determination module 204 then receives the plurality of images from the image receiving module 206. The image receiving module 206 receives the plurality of captured images from the camera(s) 106. The plurality of images captured by the camera(s) 106 may include an RGB image sequence captured by the RGB camera, and a depth image sequence captured by the depth camera while the person 102 is performing the movement.

In an example, the plurality of images may be captured in time intervals. The time intervals correspond to a gap taken by the camera(s) 106 for capturing the images. For instance, the time intervals may be 0.5 seconds, 1 second, 1.5 seconds, 2 seconds, and the like. The intervals are set by default by the processor 110 and may later be changed by the person 102 as per their moving capacity. For example, if the person 102 is performing fast movements, they would tend to choose a lower time interval to capture more images within a specific time duration. If the person 102 is performing slower movements, they may choose a higher time interval.

Once the plurality of images (RGB image sequence and depth image sequence) is received at the respective locations (first location and subsequent locations) and time intervals, the skeletal location determination module 204 extracts one or more skeletal locations of the person 102 from the image sequences. In an embodiment, the skeletal location determination module 204 extracts the skeletal locations by first down sampling the received images. Down sampling refers to a process in which the number of pixels in the captured image sequence is reduced. The down sampled images are then fed to a convolutional neural network (CNN), which includes trained data to identify the skeletal locations of the person 102. The CNN identifies/extracts the skeletal locations of the person 102 by matching the down sampled images with sample skeletal images to obtain the correct match. Once the skeletal locations are determined, they are presented to the person 102 on the display 112.

In an embodiment, the kinematics measurement module 208 determines one or more kinematic measurements of the determined one or more skeletal locations by the skeletal location determination module 204. The kinematics measurements to be determined vary from sport or movement that the person 102 is performing. The kinematic measurements involve the measurement/analysis of position, velocity, angle, acceleration, and the like at the one or more skeletal locations of the person 102. The kinematics measurement module 208 obtains the one or more kinematic measurements at the first location and then at the one or more subsequent locations, where the first location and one or more subsequent locations are received by the skeletal location determination module 204.

The kinematics measurement module 208 determines the one or more kinematic measurements at the first location and subsequent locations with the help of a plurality of markers, a light generator, and the sensor(s) 106A in-built inside the camera(s) 106. In an example, the plurality of markers may include UV markers, an optically reflective surface (for example adhesive tape), an accelerometer, and the like. The plurality of markers is secured to the joints, limbs, or skeletal locations of the person 102. Further, the number of markers may be chosen by the person 102 depending on the movement being performed.

The light generator may be embedded within the camera(s) 106 (not shown). The light generator projects light onto the plurality of markers at the first location and the one or more subsequent locations while the person 102 is performing the movement. In an example, the light may be ultra-violet (UV) light, infrared light, and the like. The sensor(s) 106A receive the light reflected towards the camera(s) 106 by each marker secured to the person 102. Different sensors are used for measuring different kinematics.

In an example, the change in the position of the skeletal locations between the first location and subsequent locations is obtained by calculating a pixel difference. The kinematics measurement module 208 divides the images into rows and columns of pixels and accordingly determines pixel coordinates for each skeletal location or location to which each marker is secured. The kinematics measurement module 208 first determines the coordinates of each skeletal location or marker location at the image(s) captured at the first location (before the person 102 performs the movement). The kinematics measurement module 208 then determines the coordinates of the same skeletal locations or marker locations for the image(s) captured at the subsequent locations. The kinematics measurement module 208 then calculates a difference/distance between the coordinates obtained at the first location and the coordinates obtained at the subsequent locations for each skeletal location or marker location. The difference/distance between the coordinates indicates a current position or difference in positions of the skeletal locations between each location of the person 102 captured by the camera(s) 106.

The kinematic measurement of angular momentum may be determined using a gyroscope sensor, the kinematic measurement for direction may be determined using a magnetometer, and the kinematic measurement of acceleration may be determined using an accelerometer. These sensors may be attached to the one or more skeletal locations or one or more body parts of the person 102. Once the kinematic measurements at the one or more skeletal locations of the person 102 are determined, they may then be presented on the display 112. The person 102 may also view the determined kinematic measurements on the movement analyzing application in their respective device 104. The person 102 may view the kinematic measurements on the display 112 while they are performing their movement or after they have performed their movement.

In an embodiment, the comparison module 210 compares the determined one or more kinematic measurements of the person 102 obtained by the kinematics measurement module 208 with an ideal kinematic movement for the person 102. The ideal kinematic movement corresponds to an appropriate level or a desired level that the person 102 is expected to perform to reach better and higher standards. The ideal kinematic movement is based on at least one of a size, a weight, an age, a gender, a limb length, a limb diameter, a limb density, a body diameter, a body density, a skeletal makeup of the person 102, and the like. The skeletal makeup of the person 102 may correspond to one or more of the sizes and densities of their body parts, such as their legs, arm, shoulders, head, limbs, and the like.

The one or more parameters size, weight, age, gender, skeletal makeup, and the like are provided by the person 102 at the time of registration with the movement analyzing application. These details are stored in the database 226. Based on these parameters, the comparison module 210 computes an ideal form for the person 102. Based on the determined ideal form, the comparison module 210 compares the kinematic measurements of the person 102 with the kinematic measurements of others having ideal forms within a similar range of the person 102 for a similar movement activity. In an example, the others may be people registered in the movement analyzing application. The comparison between the person 102 and others is displayed to the person 102 on their device 104 or on the display 112 in tabular format. For example, if the movement performed by the person 102 is weightlifting, the person may view their kinematic measurement values such as knee speed, hand speed with the knee speed and hand speed of others who had weightlifting kinematic measurements analyzed. Thus, the person 102 may be able to see how they measure when compared to movements of other people. This may help improve their form, mobility, strength, and overall performance.

In another example, the others may also be professional athletes, coaches, or professional/Olympic standards. The comparison module 210 may obtain kinematic measurements of professional athletes, coaches, or professional standards/Olympic standards using application programming interfaces (APIs), such as remote APIs, web APIs, and the like. The comparison between the person 102 and professional athletes, coaches, or professional standards/Olympic standards is also presented to the person 102 in a tabular format.

In an embodiment, the analysis module 212 provides an analysis of the person 102 based on the comparison analyzed/evaluated by the comparison module 210. In an example, the analysis may include image(s) of the person 102 while performing the movements captured by the camera(s) 106, the determined kinematic measurements of the person 102 while performing the movement, the comparison between the person 102 with others (in tabular format), and one or more suggestions provided for improvement. The analysis may be presented to the person 102 on their respective device 104 or display 112.

The one or more suggestions may be presented to the person 102 using at least one of images, text, or voice communication. The images show the correct position/form that the person 102 is expected to execute while performing the movement. The text provides written recommendations/suggestions for the person 102. For example, the written suggestions may be ‘bent left knee further down’, ‘increase left foot speed’, ‘increase hip acceleration while swinging’, and the like. The voice communication provides the suggestions using voice messages in which the suggestions are spoken out to the person 102. The suggestions may be spoken out to the person 102 while performing the movement or after performing the movement.

In an embodiment, the object location determination module 214 determines one or more location points of an object associated with the person 102 while performing the movement. In an example, the object may correspond to the equipment used by the person 102 while performing the movement or the projectile being used while performing the movement. The equipment may include a tennis racket, hockey stick, cricket bat, shuttle, a golf club, table tennis paddle, weight bar, baseball bat, and the like. The projectile may include a tennis ball, hockey puck, cricket ball, shuttlecock, golf ball, football, baseball, shot, disc, weightlifting equipment including weights and bars, and the like.

The object location determination module 214 obtains the one of more location points of the object at a first object location and then at one or more subsequent object locations. The first object location may correspond to the position of the object before it starts moving or at the start of the movement/exercise/activity. The one or more subsequent object locations correspond to the position of the object once the person 102 starts performing their movement. For example, if the person 102 is playing tennis, the first object location may correspond to the position/location of the tennis ball before it hits the person’s 102 racket. The one or more subsequent object locations may correspond to positions/locations of the tennis ball as it leaves the person’s 102 racket and crosses the tennis court.

The object location determination module 214 then receives the plurality of images from the image receiving module 206, which receives the plurality of images captured by the camera(s) 106. The plurality of images captured by the camera(s) 106 may include an object RGB image sequence captured by the RGB camera and an object depth image sequence captured by the depth camera while the person 102 is performing the movement using the object. Further, the object RGB image sequence and object depth image sequence may be captured by the camera(s) 106 in intervals. The intervals are set by default by the processor 110 and may later be changed by the person 102 as per their movement activity.

Once the plurality of images (object RGB image sequence and object depth image sequence) are received at the respective location (first object location and subsequent object locations) and time intervals, the object location determination module 214 extracts one or more object locations of the object from the object image sequences. In an embodiment, the object location determination module 214 extracts the object locations by performing a down sampling process on the received images. The down sampled object images are then fed to the CNN, which includes trained data to identify the locations of the object. The CNN identifies/extracts the locations of the object by matching the down sampled images with sample object images for obtaining the correct match. Once the one or more object locations are determined, they are presented to the person 102 on the display 112.

In an embodiment, the object kinematics measurement module 216 determines one or more kinematic measurements of the determined one or more object locations by the object location determination module 214. The kinematics measurements vary based on the movement performed by the person 102. In an example, the object kinematic measurements may include speed, acceleration, angles, rotations per minute (RPM), roll angle, and the like. The object kinematics measurement module 216 obtains the one or more object kinematic measurements at the first object location and then at the one or more subsequent object locations, where the first object location and one or more subsequent object locations are received by the object location determination module 214.

The object kinematics measurement module 216 may determine the one or more kinematic measurements at the first object location and subsequent object locations using the plurality of markers, light generator, and sensor(s)106A. The plurality of markers is secured to the object associated with or in contact with the person 102 while performing the movement. Further, the number of markers secured to the object(s) may be chosen by the person 102 depending on the movement being performed by them.

The light generator may be embedded within the camera(s) 106 (not shown), where the light generator projects light onto the plurality of markers at the first object location and the one or more subsequent object locations while the object is moving. In an example, the light generator may project UV light, infrared light, and the like. The sensor(s) 106A receive the light reflected towards the camera(s) 106 by each marker secured to the object. In an example, the change in the positions/locations of the object between the first object location and subsequent object locations is obtained by calculating a pixel difference between the captured images at each location, velocity and speed may be determined using one or more sensor measurements that are correlated to speed/velocity, the kinematic measurement angle may be determined using a gyroscope sensor, and the kinematic measurement acceleration may be determined using an accelerometer. These sensors may be attached to the one or more body parts of the person 102 and/or the object associated with the person 102 while performing the movement. In various embodiments, the sensors allow the person or object to be tracked when the person or object moves out of frame of the camera.

Additional movement sensors may be coupled to the camera to track movement of the camera relative to the person. In an exemplary embodiment, any camera movement is accounted for when the kinematic measurements are made. For example, a rotation of the camera during a movement may be measured and taken into account so that the change of position of the person(s) or object(s) in images does not affect the kinematic measurements for the person or object. Examples of the sensors on the camera that may be used to provide motion measurements of the camera relative to the moveable entity include an accelerometer, gyroscope, and/or magnetometer. Motion of the camera may also be determined based on images of the surroundings. For instance, the movement of stationary objects may be interpreted as movement by the camera. In an exemplary embodiment, camera movement is determined based movement of the background of a set of images.

Once the kinematic measurements at the one or more object locations are determined, they are then presented on the display 112. The person 102 performing the movement may also view the determined object kinematic measurements on the movement analyzing application installed in their device 104. Further, the person 102 may view the kinematic measurements of the object(s) on the display 112 while they are performing their movement or after they have performed their movement.

In an embodiment, the input receiving module 218 receives one or more inputs from the person 102 based on the analysis provided by the analysis module 212. In case the person 102 has queries, doubts, or feedback regarding the analysis presented to them, they may raise their issues. The issues may be raised while the person is performing the movement or after the person has performed the movement. The person 102 may provide their views, feedback, or opinions for the analysis presented to them using at least one of a voice message, verbal message, email message, QR code, gesture signal, and the like.

With the click of a button (not shown) on the movement analyzing application, the person 102 may provide their voice message, verbal message, and email message. The term “button”, as used herein, may refer to any touch input that, when touched, causes the application to execute a command. Examples of the button include but are not limited to a mechanical button, digital button, capacitive sensing button, inductance sensing button, and holographic button. Upon clicking the button, the person 102 may provide their feedback. For example, if the suggestion provided by the analysis module 212 is ‘increase spacing between the left and right legs’, the person 102 may replay back via the input receiving module 218 asking ‘Between what range to increase my leg spacing?’. The input receiving module 218 can receive the feedback in real-time and providing instant answers to the person’s 102 queries. The person 102 may also choose which message type they would like to use for providing their feedback upon clicking the button.

For voice message, the device 104 includes an in-built speaker and microphone, to which the person 102 may speak. Once the person 102 provides their feedback/query, the input receiving module 218 first transcribes their input speech into text, extracts keywords from the transcribed text, and then uses trained data to provide a voice answer back to the person 102. The voice answer may also be displayed on the display 112 for the person to read while listening.

For verbal messages, a chat box is presented to the person 102 on their device 104 where they may type in their feedback/query. Here, the input receiving module 218 may function as a chat bot, which is a developed program that can have conversations with people. The chatbot is trained using machine learning (ML) algorithms and is programmed to understand questions and search for the answer in a knowledge database. The chat bot may further learn from its previous and current interactions with people for developing correct answers. Further, the person 102 may also choose to embed the verbal messages into a QR code, which can be accessible upon scan. For email messages, the person 102 sends their feedback/queries to an assistance email ID of the movement analyzing application. The input receiving module 218 replies to the person 102 on their registered email ID stored in the database 226.

Further, the input receiving module 218 is capable of monitoring one or more gestures of the person 102 based on the analysis provided to them. In an example, the one or more gestures analyzed may include facial gestures and hand gestures. The camera(s) 106 captures the gestures of the person 102 after the analysis has been provided to them and communicates the captured gesture images to the input receiving module 218. The input receiving module 218 then feeds the captured gestured images to an AI engine that includes trained data to identify the correct gesture of the person 102. The AI engine identifies the appropriate gesture of the person 102 by matching the captured gesture images with sample gesture images to obtain the correct gesture. Once the proper gestures are determined, the person 102 is prompted on their device 104 or display 112 if they have any queries based on the analysis provided. For example, if the gesture captured by the AI engine is of a confused face, the AI engine may then communicate a message to the device 104 or display 112 stating, ‘Are the suggestions clear?’ or ‘Do you have any questions/doubts?’. The person 102 may then use the voice message, verbal message, or email message to provide their questions/doubts/feedback.

Further, the AI engine may also be capable of identifying the facial gestures of the person 102 while they are performing the movement. The one or more facial images captured by the camera(s) 106 of the person 102 while moving are fed to the AI engine, which identifies the appropriate gesture. Based on the identified gesture, the device 104 or display 112 may then prompt messages for the person 102. For example, if the gesture captured by the AI engine is of a happy face, the AI engine may then communicate a message to the device 104 or display 112 stating, ‘Good Work’, ‘Nice’, ‘Keep it Up’, and the like while the person 102 is performing the movement or after the person 102 has performed the movement. Such messages will increase the confidence level of the person 102 and will also help them build trust with the system 100.

In an embodiment, the video feed generation module 220 is configured to generate one or more video feeds of the person 102 and object while they are performing the movement based on the plurality of images captured by the camera(s) 106 at the one or more skeletal locations and object locations. The person 102 may select one or more captured images by the camera(s) 106 to add to the video feed. Once the images are selected, the person 102 selects an order in which they would want the images to appear and intervals for which they would want each image to stay or be displayed before transitioning to the next image(s). Further, the person 102 may also choose to customize the video feed by adding text, music, and other content.

Further, as the video feed is being played on the device 104 or displayed on the display 112, the person 102 may also be able to see their determined kinematic measurements at each skeletal location and object location alongside their image being projected on the video feed. The person 102 may also be able to view the comparison between themselves and their ideal kinematic movement (in tabular format) while the video is being played. The kinematic measurement being displayed may vary as the images are being transitioned while the video is playing. Thus, the person 102 may be able to clearly visualize their kinematic performance during their movement cycle captured by the camera(s) 106.

In an embodiment, the rating module 222 is configured to rate the person 102 based on a trend captured based on an analysis of at least two or more movements performed by the person 102. In an example, the trend may correspond to a change or shift between two or more movements made by the person 102 while performing the movement. The rating may be provided based on the ideal kinematic movement parameters analyzed by the comparison module 210. The rating may be presented to the person 102 using one or more scales, such as a star rating scale, numerical rating scale, graphical rating scale, descriptive rating scale, and the like.

In an embodiment, the recommendation module 224 is configured to recommend one or more programs, coaches, and the like for the person 102 based on the information provided in their profile received by the profile receiving module 202. The programs may correspond to one or more sports, athletic activities, or other motion-related actions suitable for the person 102 to perform based on details available in their profile that have been analyzed by the recommendation module 224. The recommendation module 224 may perform such analysis using a decision matrix. The recommendations are then presented to the person 102 using at least one of a voice message, verbal message, or email message.

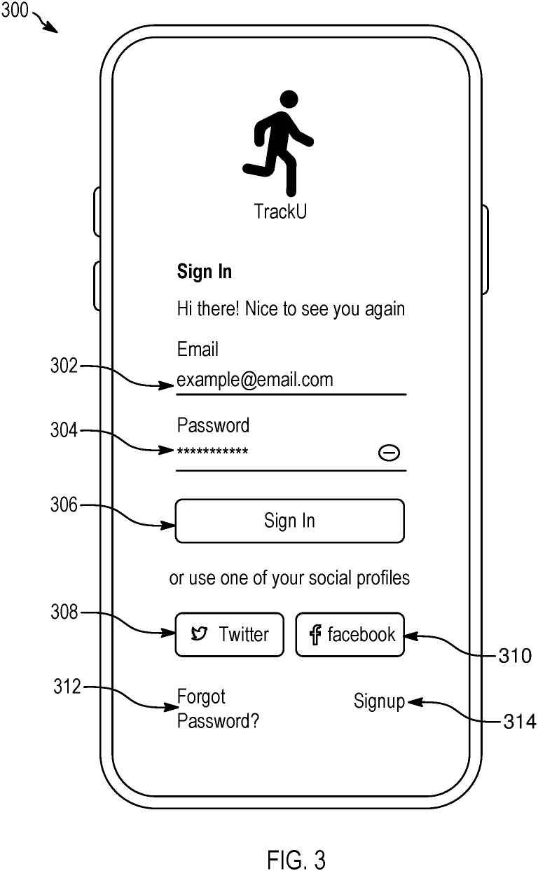

FIG. 3 illustrates a sign-in screen 300 in which the person 102 logs in to the movement analyzing application installed in their respective device 104 by providing their credentials according to an embodiment herein. To sign into the application, the person 102 provides their email 302 and password 304 as their login credentials. Once the email 302 and password 304 have been entered, the person 102 is to click the sign in button 306. In case the login credentials provided by the person 102 are incorrect, a message will instantly pop up on the sign-in screen 300 and the person 102 may re-enter the current email 302 and password 304.

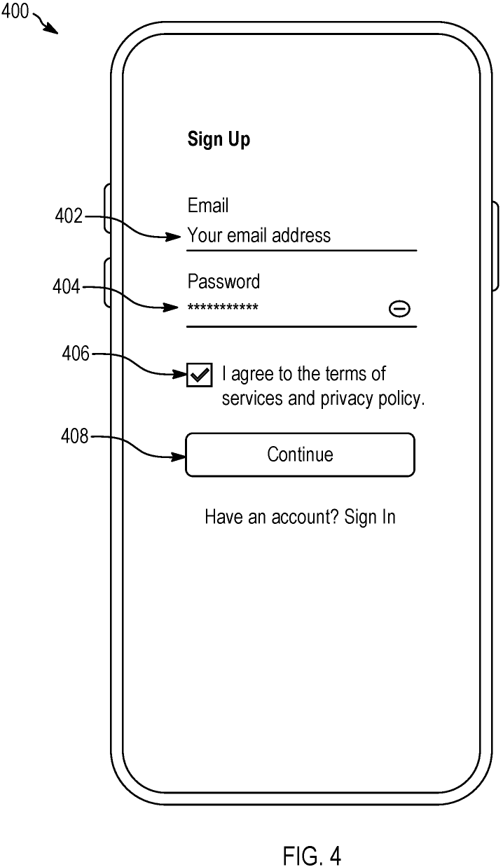

Further, the person 102 may also login using their Twitter account by clicking the Twitter button 308 and their Facebook account by clicking the Facebook button 310. The person 102 provides their Twitter/Facebook login credentials upon clicking the respective buttons 308, 310. In case the person 102 has forgotten their login credentials, they click on the forgot password link 312. Upon clicking on the forgot password link 312, the person 102 provides their new password after few security authentications. For example, the security authentications may be answer to security question(s), fingerprint verification, and the like. This is to prevent possibilities of frauds. In case the person 102 does not have an account, they click on the signup link 314, which will redirect them to the sign-up screen 400 shown in FIG. 4.

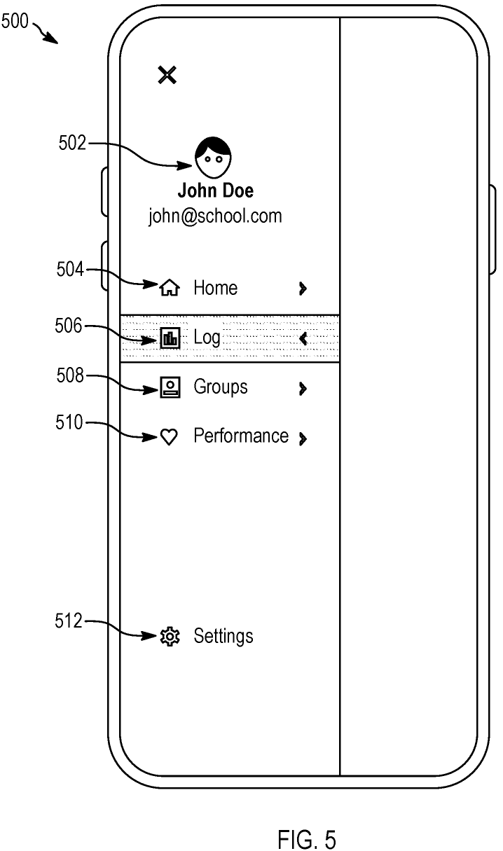

FIG. 4 illustrates the sign-up screen 400 in which the person 102 creates an account in the movement analyzing application according to an embodiment herein. The person 102 enters their email 402 and password 404. The password 404 set by the person 102 may be at least eight characters long and include a combination of letters, numbers, and symbols. In case the password is too short or not strong enough, the system 100 will prompt the person 102 to modify the password further to make it stronger. Upon entering the email 402 and password 404, the person agrees to the terms of services and privacy policy by ticking the box 406. Upon ticking the box 406, the person 102 clicks on the continue button 408. Further, the system 100 may send a one-time password (OTP) to the email 402 provided by the person 102 for verification purposes before the person 102 can start using the application. This is mainly to prevent the possibility of fraudulent account creation. Upon clicking the continue button 408, the main screen 500 as shown in FIG. 5 is presented to them.

FIG. 5 illustrates the main screen 500 of the movement analyzing application upon login according to an embodiment therein. As shown, the main screen 500 includes a profile area 502, a home option 504, a log option 506, groups option 508, a performance option 510, and a setting button 512. The profile area 502 includes a profile photo of the person 102 along with their email address. Upon clicking the log option 508, the person 102 may view a history of their movements along with the one or more kinematic measurements captured for each movement. Upon clicking the groups option 508, the person 102 may view details of one or more groups they are a part of. Upon clicking the performance option 510, the person 102 may view a performance summary of a movement performed by them.

Upon clicking the settings button 512, the person 102 may be able to control one or more parameters of the device 104 in which the movement analyzing application is installed. In an example, the one or more parameters may be display, sound, notifications, storage space, and the like.

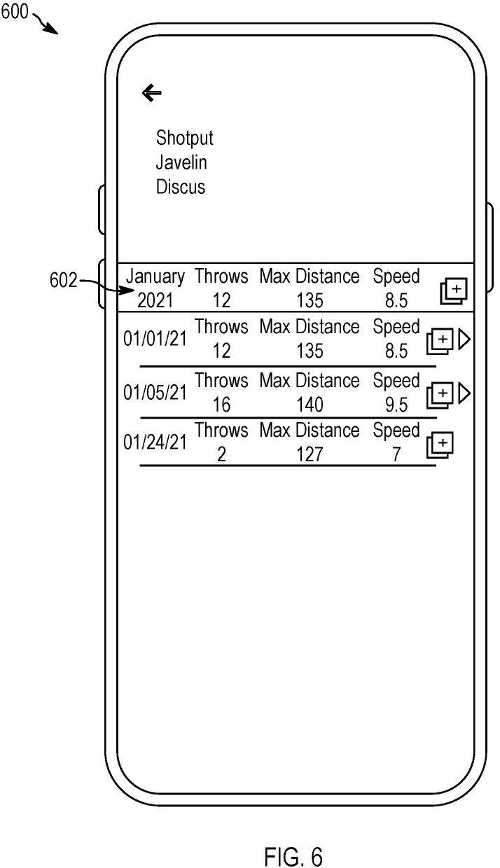

FIG. 6 illustrates a log screen 600 of the movement analyzing application upon clicking the log option 506 of the main screen 500 in FIG. 5 according to an embodiment herein. As shown, the log screen 600 includes a performance log 602 of the person 102 while performing one or more movements. The movement shown in the log screen 600 is for athletic related activities such as shotput, javelin, and discus. The performance log 602 includes the date on which the movement was performed by the person 102 along with one or more kinematic measurements such as throws, maximum distance, speed of the person 102 or object determined by the kinematic measurements module 208, 216.

Thus, the performance log 602 summarizes a history/timeline of the movements made by the person 102 and the respective kinematic measurements. For example (as shown in FIG. 6), on Jan. 1, 2021, the number of throws was 12, the maximum distance was 135 feet, and the speed was 8.5 km/hr. On Jan. 5, 2021, the number of throws was 16, the maximum distance was 140 feet, and the speed was 9.5 km/hr. On Jan. 24, 2021, the number of throws was 2, the maximum distance was 127 feet, and the speed was 7 km/hr.

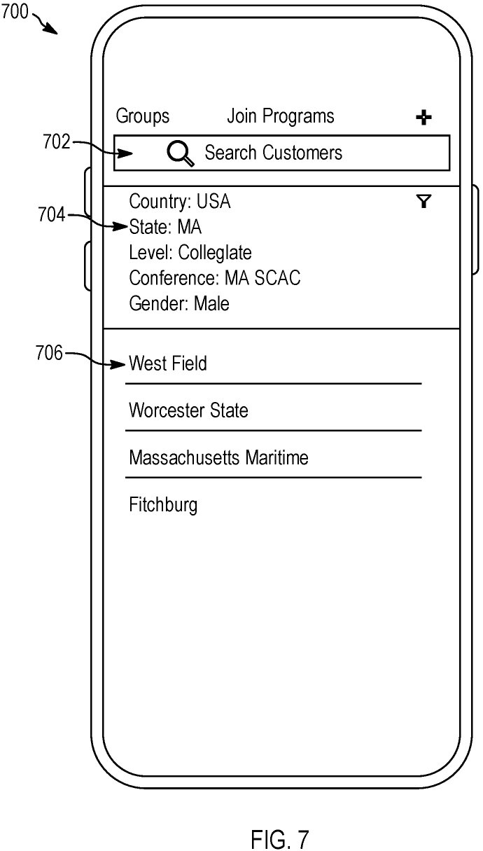

FIG. 7 illustrates a group screen 700 of the movement analyzing application upon clicking the groups option 508 of the main screen 500 in FIG. 5 according to an embodiment herein. As shown, the group screen 700 includes a search box 702, selected group area 704, and other group area 706.

The person 102 may search for one or more additional groups/clubs/leagues to join or do research about by typing in the search box 702. The person 102 may type the group name, locations, cities, and the like and the search box 702 will present a filtered set of groups/clubs/leagues to the person 102 for viewing. The person 102 may choose to join the filtered set of groups/clubs/leagues mentioned to them by clicking on an apply button (not shown). The person 102 may then provide their personal and athletic-related information. The selected group area 704 includes information about the person 102. For example, the information may include the country, state, level, conference information, and gender associated with the person 102. Further, the other group area 706 shows the groups/clubs/leagues that the person 102 is already a part of.

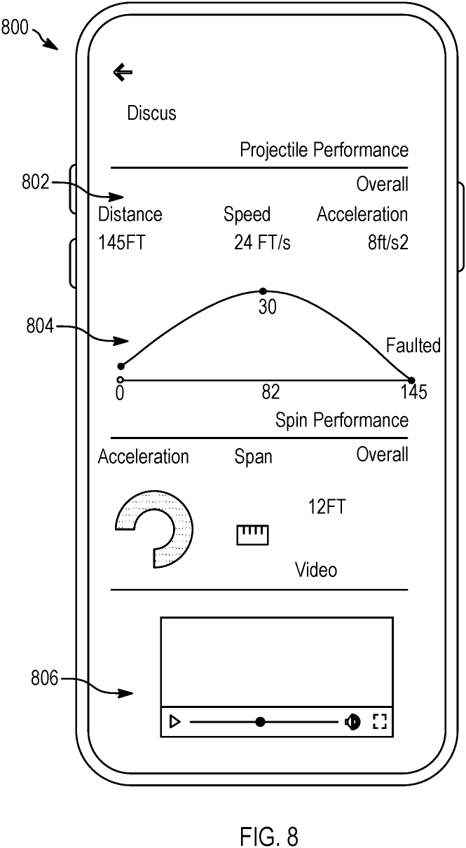

FIG. 8 illustrates a performance screen 800 of the movement analyzing application upon clicking the performance option 510 of the main screen 500 in FIG. 5 according to an embodiment herein. As shown, the performance screen 800 includes a text area 802, a graph area 804, and a video area 806. The performance screen 800 shown in FIG. 8 is illustrating the performance of the projectile used by the person 102 while performing a discus movement/activity. In an example, the projectile corresponds to a disc.

The text area 802 includes one or more kinematic measurements of the projectile determined by the object kinematics measurement module 214. The kinematic measurements of the disc displayed in the text area 802 are distance, speed, and acceleration. As shown, the distance is 145 FT, the speed is 25 FT/s, and the acceleration is 8 ft/s². The graph area 804 shows a graph of the projectile from its start position at zero feet to its end position at 145 feet. The graph also shows the height (which is 30 feet) that the projectile reached during its movement. Further, the video area 806 shows a video generated by the video feed generation module 220 to be played by the person 102. The generated video incudes the plurality of images captured by the camera(s) 106 at the one or more skeletal locations of the person 102 and one or more object locations of the projectile while the person 102 is performing the discus movement. This generated video may also be downloaded and stored in a memory of the device 104 or shared in social media (Facebook, Instagram) profiles/pages associated with the person 102.

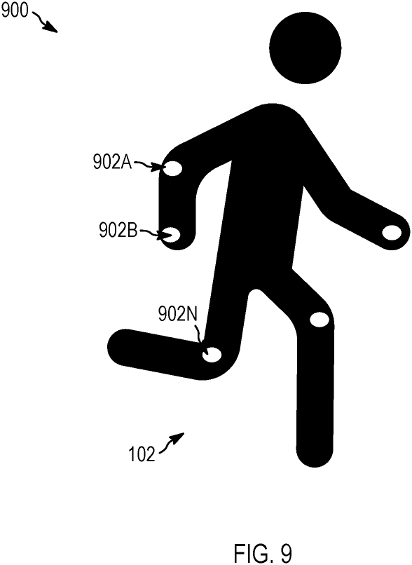

FIG. 9 illustrates a view 900 of the person 102 in which a plurality of markers 902A, 902B, and 902N are secured to their body parts according to an embodiment herein. The plurality of markers may be secured to one or more body parts of the person 102, such as ankles, knees, hip, wrists, elbows, shoulders, and the like. In an example, the plurality of markers may include infrared markers, UV markers, optically reflective surfaces, adhesive tape, and the like. Further, the plurality of markers may be of a circular shape, square shape, triangular shape, hexagonal shape, and the like. The plurality of markers may be manufactured from formulated material, which reflects one or more wavelengths illuminated by the light source.

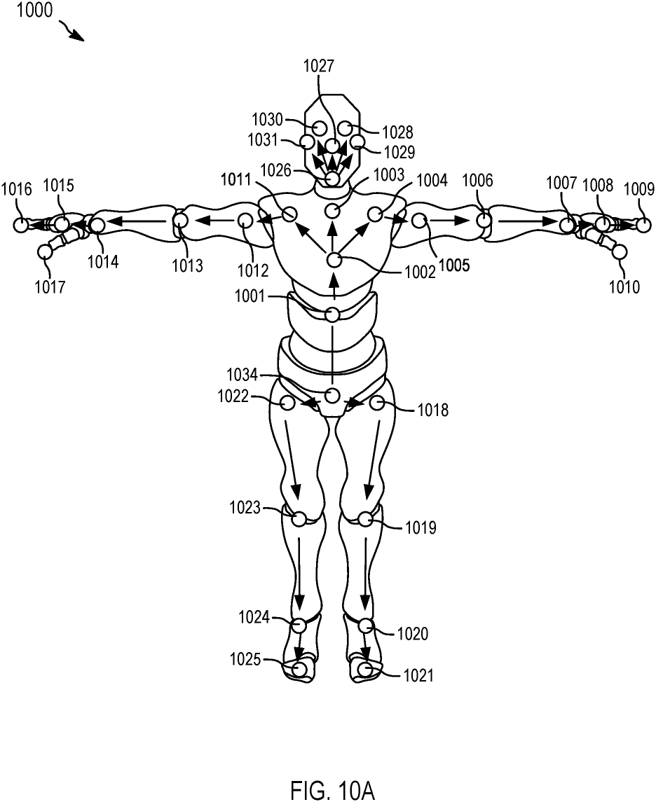

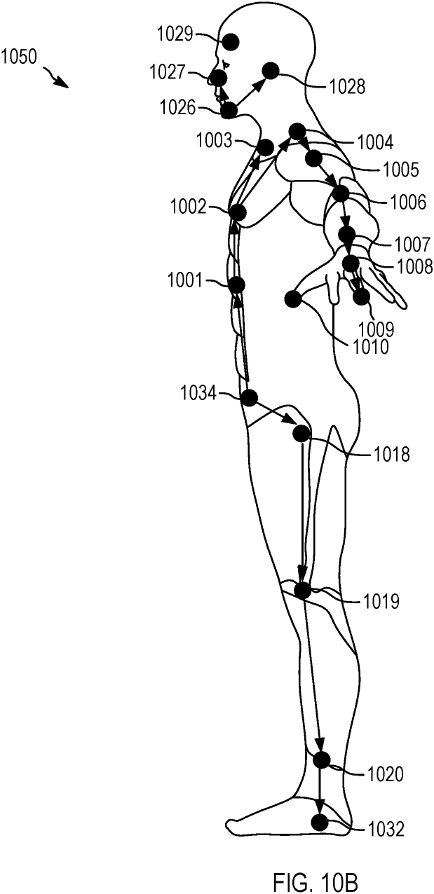

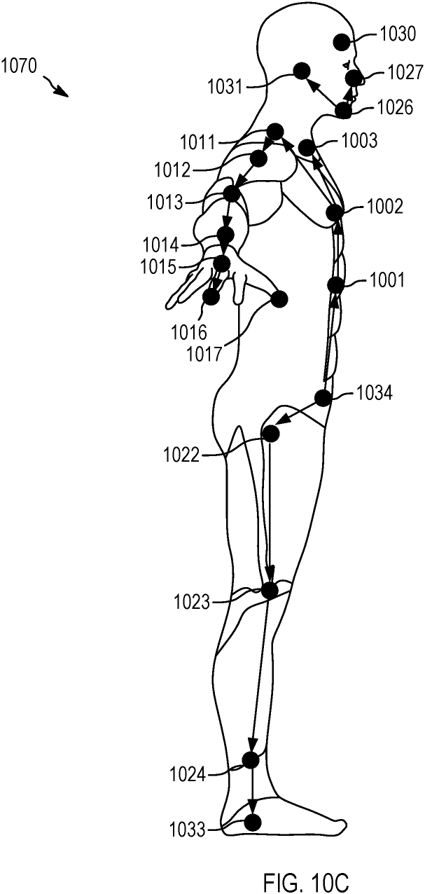

FIGS. 10A-10C, FIG. 10A illustrates a view 1000 of the person 102 in which one or more skeletal locations are shown according to an embodiment herein. FIG. 10B illustrates a left-side view 1050 of the one or more skeletal locations shown in FIG. 10A as applied to a human individual. FIG. 10C illustrates a right-side view 1070 of the one or more skeletal locations shown in FIG. 10A as applied to a human individual. In an exemplary embodiment, the position for each of the skeletal locations comprise a three-dimensional spatial coordinate and the three dimensional rotational coordinate.

The view 1000 of the person 102 includes a total of thirty-four skeletal locations referenced by numbers mentioned in the table below:

| Skeletal Index | Skeletal Location Name |
|---|---|
| 1001 | Naval Spine |
| 1002 | Chest Spine |
| 1003 | Neck |
| 1004 | Left Clavicle |
| 1005 | Left Shoulder |
| 1006 | Left Elbow |
| 1007 | Left Wrist |
| 1008 | Left Hand |
| 1009 | Left Hand Tip |
| 1010 | Left Thumb |
| 1011 | Right Clavicle |
| 1012 | Right Shoulder |
| 1013 | Right Elbow |
| 1014 | Right Wrist |
| 1015 | Right Hand |
| 1016 | Right Hand Tip |
| 1017 | Right Thumb |
| 1018 | Left Hip |
| 1019 | Left Knee |
| 1020 | Left Ankle |
| 1021 | Left Foot |
| 1022 | Right Hip |
| 1023 | Right Knee |
| 1024 | Right Ankle |
| 1025 | Right Foot |
| 1026 | Head |
| 1027 | Nose |
| 1028 | Left Eye |
| 1029 | Left Ear |
| 1030 | Right Eye |
| 1031 | Right Ear |
| 1032 | Left Heel |
| 1033 | Right Heel |
| 1034 | Pelvic |

Arrows in FIG. 10A indicate a parent-child relationship between two skeletal locations. For instance, the naval spine 1001 skeletal location is a parent node to the chest spine 1002 skeletal location. In various embodiments, coordinates of the skeletal locations may be positioned at different locations in or around the human body. In an exemplary embodiment, the skeletal locations are determined procedurally by the skeletal location determination module 204 based on the images of the person. In various embodiments, the person may be represented by different numbers of skeletal locations which may be positioned differently. For instance, the skeletal location determination module 204 may determine at set 12 skeletal locations to represent a person.

In various embodiments, a bone armature, such as that which is shown in FIG. 10A, may be generated to elegantly demonstrate a person’s recorded movements or movements to which to person is tasked to imitate. In an exemplary embodiment, the skeletal locations may be superimposed on a view of the person, as shown in FIGS. 10B and 10C. Skeletal locations for any two individuals will differ, however, recorded movements of skeletal locations for a first individual may be extrapolated to other individuals of different sizes based on the parent-child relationships between the skeletal locations.

As shown in the left side view of FIG. 10B, the skeletal location determination module 204 determines positions of the skeletal locations as the person moves and rotates. The skeletal location determination module 204 may ascertain the one or more positions of skeletal locations that are hidden from view. For instance, a position of the right clavicle 1011 in FIG. 10B may be estimated, even though it is hidden from view, based on its parent-child relationships with the right shoulder 1012 and chest spine 1002 and previous images of the person.

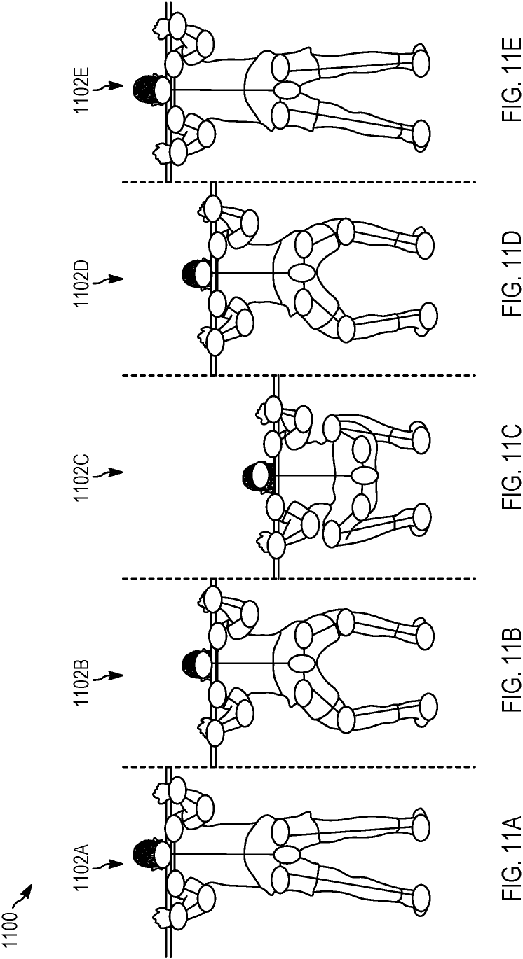

FIGS. 11A-E illustrate sequences 1100 of the person 102 captured by the camera(s) 106 while performing a weightlifting exercise according to an embodiment herein. The sequences 1100 of the person 102 are captured at a first location 1102A and then at one of more subsequent locations 1102B-E at intervals (for example 0.5 seconds, 1 second, 1.5 seconds, 2 seconds, and the like). The first location 1102A corresponds to the position/form of the person 102 at the start of the weightlifting exercise movement. In an example, the first location 1102A may be a standing position.

For example, the kinematics measurement module 208 may determine that the speed and other movement measurements are near zero while the person is in the stationary position shown in FIG. 11A. The kinematics measurement module 208 may further determine tension at the various skeletal locations and/or calculate isometric forces on the person. For instance, the analysis module 212 may determine that the person has more tension on one side if the bar is off-balance.

The one or more subsequent locations 1102B-E corresponds to positions/forms of the person 102 once they start the weightlifting exercise movement. In an example, subsequent locations 1102B and 1102B corresponds to a squat position, subsequent location 1102C corresponds to a bend position, and subsequent location 1102D corresponds to a finish position. In addition to the tension at the various skeletal locations at each of the positions, the kinematics measurement module 208 outputs speed, acceleration, rotation, angular momentum, angular acceleration, and other movement measurements as the person is performing the squat movement.

Further, the one or more skeletal locations of the person 102 are also shown on the first location 1102A and one of more subsequent locations 1102B-E image sequences, where the skeletal locations are obtained by the skeletal location determination module 204. The number of skeletal locations may vary based on the movement being performed by the person 102. In the embodiment shown in FIG. 11, there are twelve skeletal locations determined. Once the skeletal locations are determined and marked on the first location 1102A and subsequent locations 1102BE image sequences, the kinematics measurement module 208 determines kinematic measurements at the first location 1102A image sequence and the subsequent locations 1102B-E image sequences. The determined kinematic measurements may then be presented to the person 102.

The comparison module 210 may compare the kinematic measurements of the person at the various locations to one or more ideal movements. An ideal movement may be any movement that could be performed by the person given their body proportions and skeletal makeup. In an exemplary embodiment, the ideal movement may be based on a movement of a professional trainer where the ideal movement is modified to fit the skeletal proportions of the person. For example, if the professional trainer, from which the ideal movement is based, has a shorter torso and longer legs than the person, the comparison module 210 may determine an ideal movement of the squat that goes lower than professional trainer’s movement to accommodate for the change in body structure.

In various embodiments, the comparison module 210 may compare a person’s movement to more than one ideal movement. In one example, a person’s movement is compared to an advanced movement, an intermediate movement, and a beginner movement. The analysis module 212 may then direct the person to emulate the ideal forms of one of the advanced, intermediate, or beginner movements based on a skill/ability of the person.

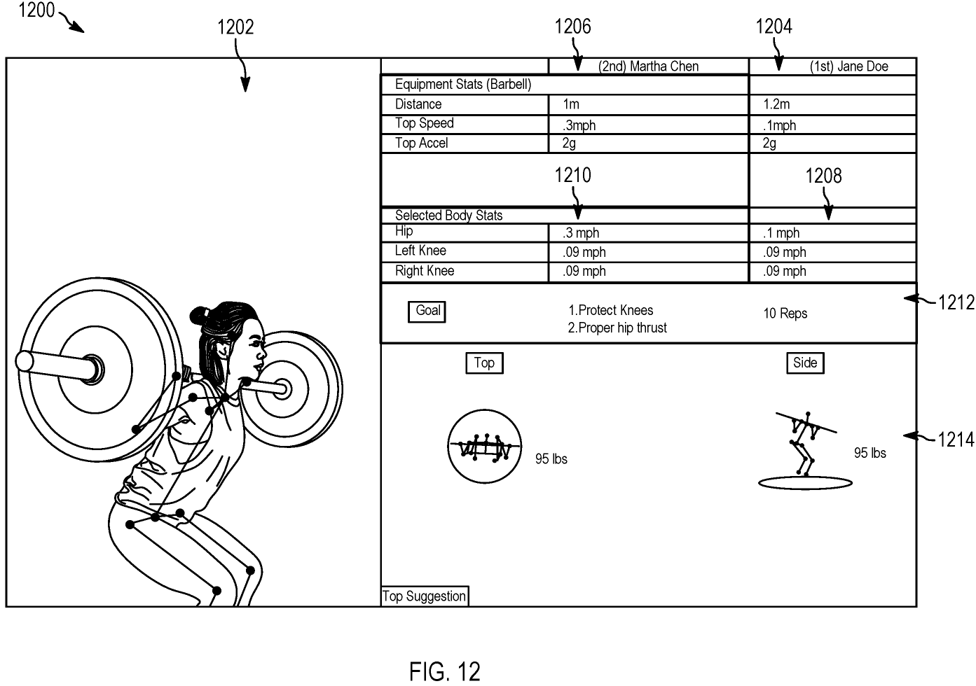

FIG. 12 illustrates an analysis view 1200 of the person 102 illustrating kinematic measurements determined for movements performed by them while weightlifting according to an embodiment herein. As shown, the analysis view 1200 includes a weightlifting image 1202, barbell kinematic stats 1204, 1206, body stats 1208, 1210, a goal area 1212, and a suggestion area 1214. The weightlifting image 1202 corresponds to an image captured by the camera(s) 106 while the person 102 is performing the weightlifting exercise. The weightlifting image 1202 also includes skeletal locations marked using black dots.

The barbell kinematic stats refer to one or more kinematic measurements obtained by the object kinematics measurement module 216 corresponding to the barbell equipment used while weightlifting. The kinematic measurements corresponding to the barbell include distance, top speed, and top acceleration. In an example, the barbel kinematic stats may include a first stats set 1204 and a second stats set 1206. The first stats set 1204 corresponds to the kinematic measurements associated with the person 102 (John Doe), and the second stats set 1206 corresponds to the kinematic measurements related to another person (Martha Chen).

The body stats 1208, 1210 refer to one or more kinematic measures obtained by the kinematics measurement module 208 corresponding to the movements made by the person 102 while weightlifting. The kinematics measurements corresponding to the person 102 includes hip speed, left knee speed, and right knee speed. In an example, the body stats 1208, 1210 may include a first body stat 1208 and a second body stat 1210. The first body stat 1208 corresponds to the kinematic measurements associated with the person 102 (John Doe) and the second body stat 1210 corresponds to the kinematic measurements related to the other person (Martha Chen). The analysis view 1200 displays and compares the kinematic measurements associated with the person 102 and the object (barbell) with the kinematic measurements of another weightlifter using a comparison table. Thus, the person 102 may be able to visualize themselves and see how well they stand.

Further, the goal area 1212 displays one or more goals that the person 102 may try to accomplish. For instance, the goals mentioned in the goal area 1212 are ‘protect knees’, ‘proper hip thrust’ and ’ 10 reps’. The suggestion area 1214 displays one or more suggestions for the person 102 to take into consideration for improving their form and achieving the goals mentioned in the goal area 1212. The suggestion area 1214 provides suggestions for the person 102 using images. The images show the correct position/form that the person 102 may try to accomplish while performing the movement.

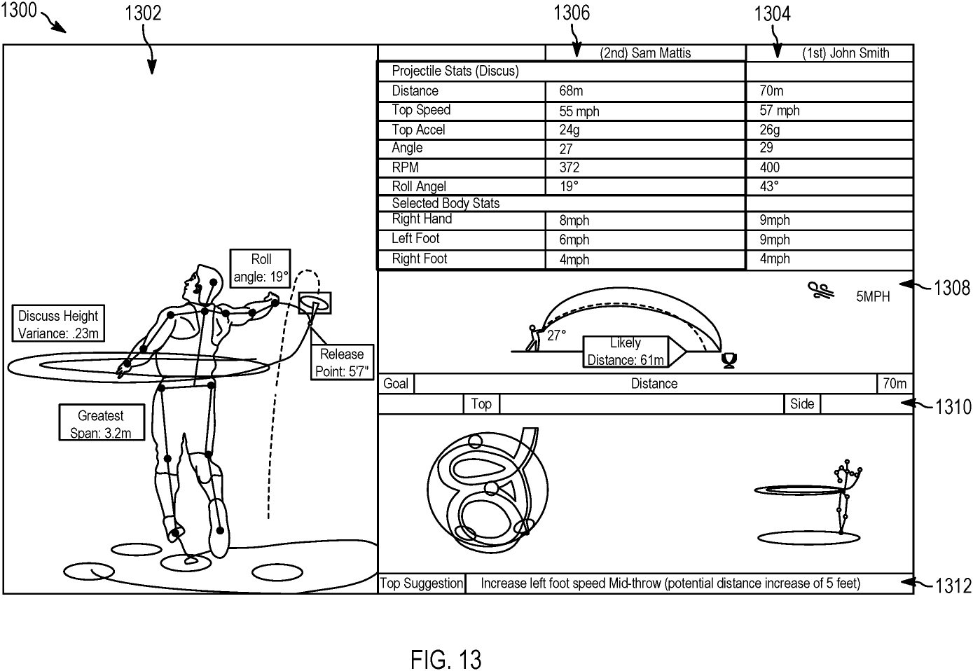

FIG. 13 illustrates an analysis view 1300 of the person 102 illustrating kinematic measurements determined for movements performed by them while playing discus according to an embodiment herein. As shown, the analysis view 1300 includes a discus image 1302, kinematic stats 1304, 1306, a disc image 1308, a goal area 1310, and a suggestion area 1312. The discus image 1302 corresponds to an image captured by the camera(s) 106 while the person 102 is throwing the discus. The discus image 1302 also includes skeletal locations marked using black dots.

The kinematic stats 1304, 1306 refer to one or more kinematic measures obtained by the object kinematics measurement module 216 corresponding to the discus projectile/object used, and the kinematics measurement module 208 corresponding to the movements made by the person 102. The kinematic measurements corresponding to the discus include distance, top speed, top acceleration, angle, RPM, and roll angle. The kinematic measurement corresponding to the person 102 includes right-hand speed, left-foot speed, and right-foot speed. For example, the kinematic stats 1304, 1306 may include a first kinematic stat 1304 and a second kinematic stat 1306. The first kinematic stat 1304 corresponds to the kinematic measurements associated with the person 102 (John Doe) and the second kinematic stat 1306 corresponds to the kinematic measurements associated with another person (Sam Mattis).

The disc image 1308 displays the image of the discus while moving from its starting point to its end point. The starting point is the hand of the person 102 and the end point is when the disc touches the ground, which is located approximately 61 meters from the person 102. The analysis view 1300 displays and compares the kinematic measurements associated with the person 102 and the object (disc) with the kinematic measurements of another discus player using a comparison table.

Further, the goal area 1310 displays one or more goals that the person 102 may try to achieve. For instance, the goals shown in the goal area 1310 sets a discus throwing goal of 70 feet. The suggestion area 1312 displays one or more suggestions for the person 102 to take into consideration for improving their form and achieving the goals mentioned in the goal area 1310. The suggestion area 1312 provides suggestions for the person 102 in writing. For instance, the suggestion mentioned in the suggestion area 1312 mentions ‘increase left foot speed mid-throw’.

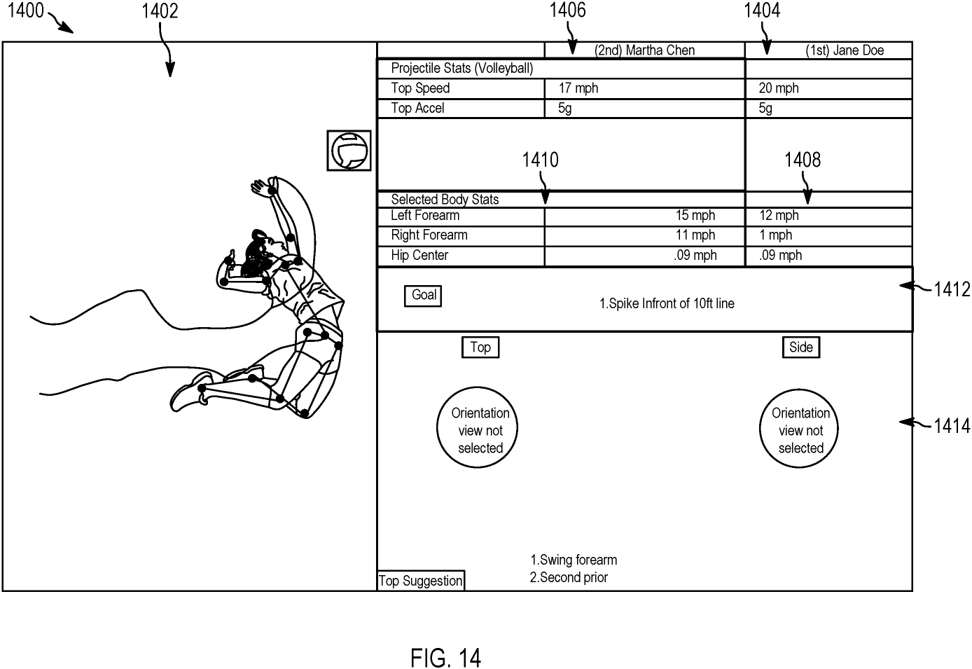

FIG. 14 illustrates an analysis view 1400 of the person 102 illustrating kinematic measurements determined for movements performed by them while playing volleyball according to an embodiment herein. As shown, the analysis view 1400 includes a volleyball image 1402, volleyball kinematic stats, body stats 1408, 1410, a goal area 1412, and a suggestion area 1414. The volleyball image 1402 corresponds to an image captured by the camera(s) 106 while the person 102 is playing volleyball. The volleyball image 1402 also includes the skeletal locations of the person 102 marked using black dots.

The volleyball kinematic stats refer to one or more kinematic measurements obtained by the object kinematics measurement module 216 corresponding to the volleyball used. The kinematic measurements corresponding to the volleyball includes top speed and top acceleration. For example, the volleyball kinematic stats may include a first stats set 1404 and a second stats set 1406. The first stats set 1404 corresponds to the kinematic measurements associated with the person 102 (Jane Doe), and the second stats set 1406 corresponds to the kinematic measurements related to another person (Martha Chen).

The body stats 1408, 1410 refer to one or more kinematic measurements obtained by the kinematics measurement module 208 corresponding to the movements made by the person 102 while paying volleyball. The kinematics measurements corresponding to the person 102 includes left forearm speed, right forearm speed, and hip center. In an example, the body stats 1408, 1410 may include a first body stat 1408 and a second body stat 1410. The first body stat 1408 corresponds to the kinematic measurement associated with the person 102 (Jane Doe) and the second body stat 1410 corresponds to the kinematic measurements related to the other person (Martha Chen). The analysis view 1400 displays and compares the kinematic measurements associated with the person 102 and the object (volleyball) with the kinematic measurements of another person using a comparison table. Thus, the person 102 may be able to visualize themselves and see how well they stand.

Further, the goal area 1412 displays one or more goals that the person 102 may try to accomplish. For instance, the goals mentioned in the goal area 1412 are ‘spike in front of 10 ft line’. The suggestion area 1414 displays one or more suggestions for the person 102 to take into consideration for improving their form and achieving the goals mentioned in the goal area 1412. The suggestion area 1414 provides suggestions for the person 102 in writing. For instance, the suggestions mentioned in the suggestion area 1414 are ‘swing forearm’ and ‘second prior’.

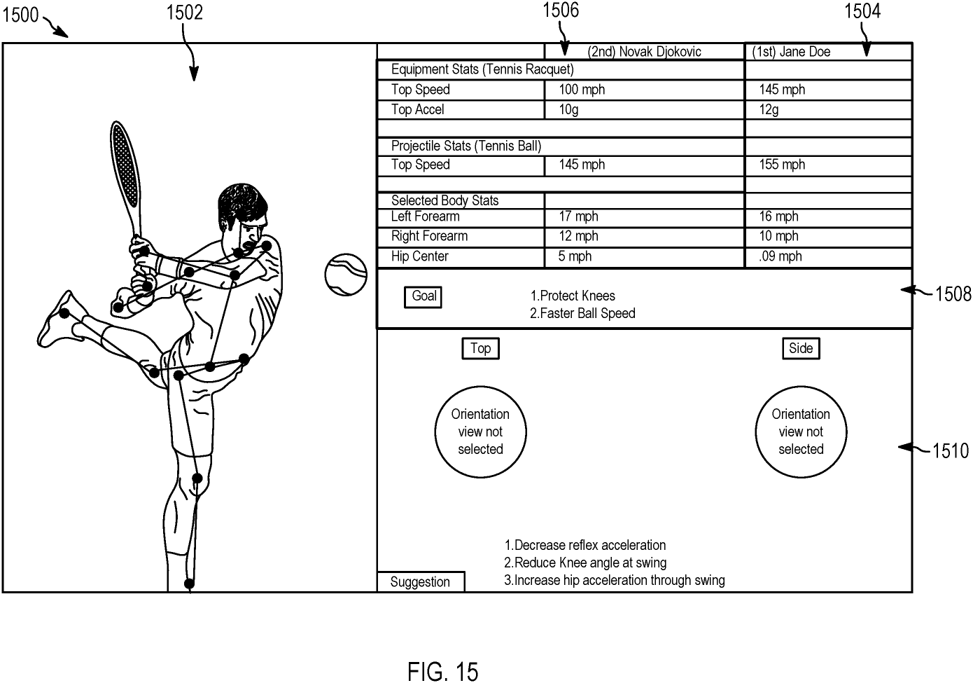

FIG. 15 illustrates an analysis view 1500 of the person 102 illustrating kinematic measurements determined for movements performed by them while playing tennis according to an embodiment herein. As shown, the analysis view 1500 includes a tennis image 1502, kinematic stats 1504, 1506, a goal area 1508, and a suggestion area 1510. The tennis image 1502 corresponds to an image captured by the camera(s) 106 while the person 102 is playing tennis. The captured tennis image 1502 also includes one or more skeletal locations of the person 102 marked using black dots.

The kinematic stats 1504, 1506 refer to one or more kinematic measures obtained by the object kinematics measurement module 216 corresponding to the objects (tennis racquet and tennis ball), and the kinematics measurement module 208 corresponding to the movements made by the person 102. The kinematic measurements corresponding to the tennis racquet include top acceleration and speed. The kinematic measurements corresponding to the tennis ball include top speed. The kinematic measurements corresponding to the person 102 include left forearm speed, right forearm speed, and hip speed. In an example, the kinematic stats 1504, 1506 may include a first kinematic stat 1504 and a second kinematic stat 1506. The first kinematic stat 1504 corresponds to the kinematic measurements associated with the person 102 (Jane Doe), and the second kinematic stat 1506 corresponds to the kinematic measurements related to another person (Novak Djokovic).

Further, the goal area 1508 displays one or more goals that the person 102 may try to achieve. For instance, the goals mentioned in the goal area 1508 are ‘protect knees’ and ‘faster ball speed.’ The suggestion area 1510 displays one or more suggestions for the person 102 to take into consideration for improving their form and achieving the goals mentioned in the goal area 1508. The suggestion area 1510 provides suggestions for the person 102 in writing. For instance, the suggestion in the suggestion area 1510 mentions ‘decrease reflux acceleration’, ‘reduce knee angle at swing’, and ‘increase hip acceleration through swing’.

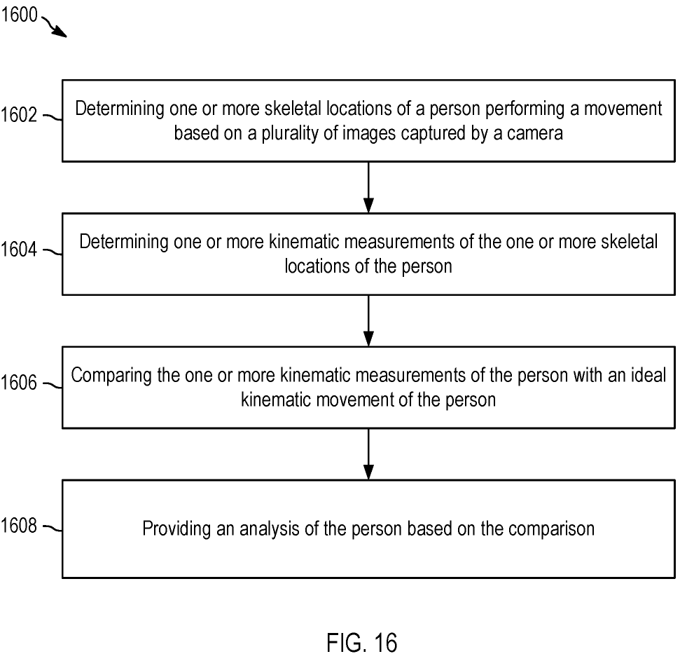

FIG. 16 illustrates a flowchart illustrating a method 1600 for analyzing one or more movements of the person 102 according to an embodiment herein. The steps 1602-1608 of the method 1600 may be executed using the processor 110 of FIGS. 1-2. Each step is explained in further detail below.

At step 1602, one or more skeletal locations of the person 102 are determined based on a plurality of images captured by the camera(s) 106. In an example, the skeletal locations may include ankle positions, knee positions, hip positions, wrist positions, elbow positions, shoulder positions, and the like. The plurality of images captured by the camera(s) 106 may include an RGB image sequence captured by the RGB camera, and a depth image sequence captured by the depth camera while the person 102 is performing the movement. The skeletal locations are determined by sampling the plurality of images captured by the camera(s) 106. The down-sampled images are then fed to a convolutional neural network (CNN), which is trained to identify the skeletal locations via trained data. The CNN recognizes/extracts the skeletal locations of the person 102 by matching the down sampled images with one or more sample skeletal photos stored in a CNN database of images to obtain the correct match.

At step 1604, one or more kinematic measurements of the one or more skeletal locations are determined. The kinematic measurements involve measuring factors such as the position, velocity, angle, acceleration, and the like at the determined one or more skeletal locations of the person 102. Further, the number of kinematic measurements varies from movement performed by the person 102.

At step 1606, the determined one or more kinematic measurements are compared with an ideal kinematic movement of the person. The ideal kinematic movement corresponds to the desired level that the person 102 is expected to perform to reach higher standards. The ideal kinematic movement is based on at least one of a size, a weight, an age, a gender, a limb diameter, a limb density, a body diameter, a body density, a skeletal makeup of the person 102, and the like.. The parameters size, weight, age, gender, and skeletal makeup are provided by the person 102, and the ideal form of the person 102 is determined based on these parameters. Based on the determined ideal form, the determined kinematic measurements of the person 102 are compared with kinematic measurements of others having ideal forms within a similar range of the person for a particular movement activity. In an example, the others may be people registered in the movement analyzing application or professional athletes, coaches, or professional/Olympic standards. The comparison between the person 102 and others is presented to the person 102 using a tabular format.

At step 1608, an analysis of the person 102 is provided based on the comparison. The analysis may be visible to the person 102 on their respective device 104 or display 112. The analysis may include one or more image(s) of the person 102 while performing the movements captured by the camera(s) 106, determined kinematic measurements of the person 102 while performing the movement, the comparison between the person 102 with others (in tabular format), and one or more suggestions/recommendations for improvement.

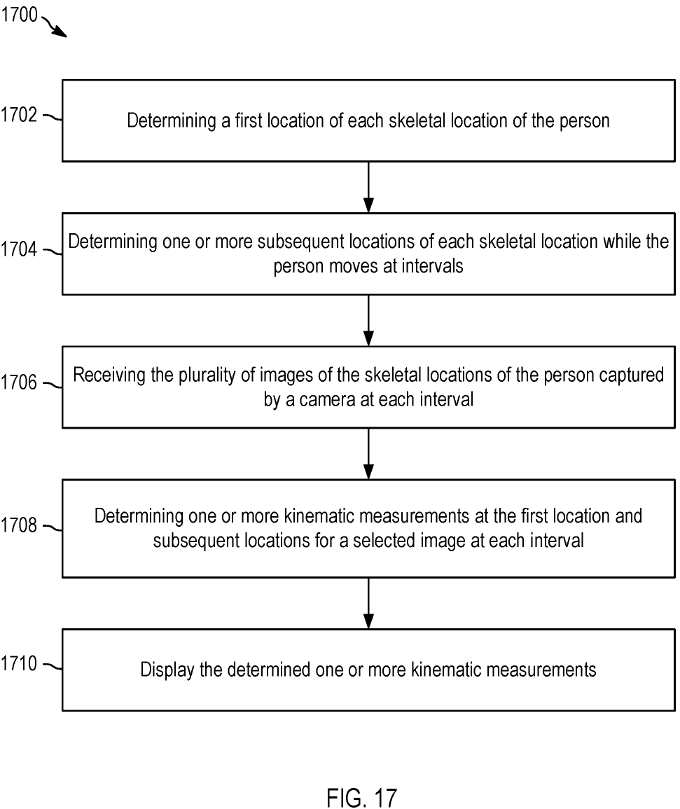

FIG. 17 illustrates a flowchart illustrating a method 1700 for displaying kinematic measurements of one or more skeletal locations of the person 102 while performing the movement at intervals according to an embodiment herein. The steps 1702-1710 of the method 1700 may be executed using the processor 110 of FIGS. 1-2. Each step is explained in further detail below.

At step 1702, a first location of each skeletal location of the person 102 is determined. The first location may correspond to a position/location of the person 102 before they start performing their respective movement activity. The first location may also correspond to a first movement performed during the movement activity.

At step 1704, one or more subsequent locations of each skeletal location is determined at intervals. The one or more subsequent locations refer to positions/locations of the person 102 once they start to perform the movement. For example, if the person 102 is performing a weightlifting exercise, the one or more subsequent locations may correspond to positions/locations of the person 102 as they are lifting the barbell from the ground until the barbell is placed above their shoulders.

At step 1706, the plurality of images of the skeletal locations of the person 102 captured by the camera(s) 106 are received at intervals. The intervals correspond to gaps taken be the camera(s) 106 while capturing each image. The intervals are set by default by the processor 110 and may later be changed by the person 102 as per the type of movement being performed.

At step 1708, one or more kinematic measurements are determined at the first location and one or more subsequent locations for a selected image at each interval. The one or more kinematic measurements may be obtained using a plurality of markers secured to the body parts of the person 102, a light generator embedded within the camera(s) 106, and the sensor(s) 106A in-built inside the camera(s) 106. The light generator projects light onto the plurality of markers at the first location and the one or more subsequent locations while the person 102 is performing the movement. The sensor(s) 106A receive the light reflected by the plurality of markers, where different sensors are used for measuring different kinematics. In an example, the change in skeletal location positions between the first location and subsequent locations is obtained by calculating a pixel difference between the images captured at the respective locations, the kinematic measurement angle may be determined using a gyroscope sensor, and the kinematic measurement acceleration may be determined using an accelerometer.

At step 1710, the determined one or more kinematic measurements are displayed on the display 112 for the person 102 to view. In an example, the person 102 may view the kinematic measurements on the display 112 while they are performing their movement or after they have performed their movement.

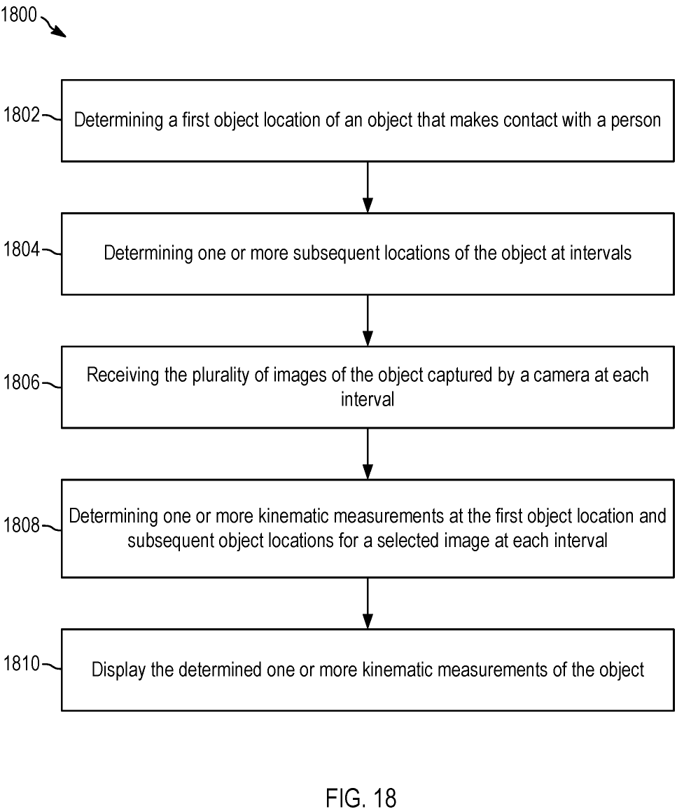

FIG. 18 illustrates a flowchart illustrating a method 1800 for displaying kinematic measurements of one or more locations of the object associated with the person 102 while performing the movement according to an embodiment herein. The steps 1802-1810 of the method 1800 may be executed using the processor 110 of FIGS. 1-2. Each step is explained in further detail below.

At step 1802, a first object location of an object associated with the person 102 or that contacts the person 102 while performing the movement is determined. In an example, the object may correspond to equipment or projectiles used by the person 102 while performing the movement. The first object location may correspond to the position of the object at the start of the movement.

At step 1804, one or more subsequent object locations of the object is determined at intervals. The one or more subsequent object locations correspond to the position of the object once the person 102 starts the movement.

At step 1806, the plurality of images of the object captured by the camera(s) 106 are received at intervals. The intervals correspond to pauses taken be the camera(s) 106 while capturing each image. The intervals are set by default by the processor 110 and may later be changed by the person 102 as per their movement activity being performed.

At step 1808, one or more kinematic measurements are determined at the first object location and subsequent object locations for a selected image at each interval. The one or more kinematic measurements at the respective object locations may be determined using the plurality of markers secured to the object, light generator embedded within the camera(s) 106, and sensor(s)106A in-built inside the camera(s) 106. The light generator projects light onto the plurality of markers at the first object location and subsequent object locations and the sensor(s) 106A receive the light reflected from each marker. The change in position of the object between the first object location and subsequent object locations is obtained by calculating a pixel difference between the captured images at each location, the kinematic measurement angle may be determined using a gyroscope sensor, and the kinematic measurement acceleration may be determined using an accelerometer.

At step 1810, the determined one or more kinematic measurements of the object are displayed on the display 112. In an example, the person 102 may view the kinematic measurements on the display 112 while they are performing their movement or after they have performed their movement.

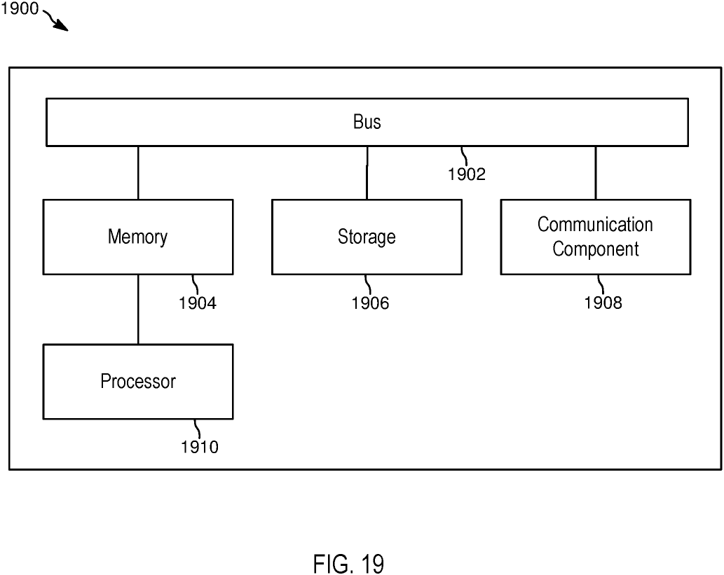

FIG. 19 is a schematic of an embodiment of a computer system 1900 that may be implemented to carry out the disclosed subject matter. As shown, the computer system 1900 includes a bus 1902, a memory 1904, a storage 1906, a communication component 1908, and a processor 1910. The bus 1902 may connect the various components of the computer system 1900. The bus 1902 may be connected to the memory 1904, which stores data that is being transmitted to the various parts of the computer system 1900 through the bus 1902. Various types of memory 1904 may be random access memory (“RAM”) and read-only memory (“ROM”). The memory 1904 may transmit instructions to the processor 1910 to be executed.

The processor 1910 may process instructions that are transmitted to the processor 1910 from the memory 1904. Executed instructions may be transmitted from the memory 1904 to the various components of the computer system 1900. Various types of processors 1910 may be central processing units (“CPUs”), graphics processing units (“GPUs”), field programmable gate arrays (“FPGAs”), complex programming logic devices (“CPLDs”), and application specific integrated circuits (“ASICs”). The processor 1910 may execute instructions that are passed to the processor 1910 by the client user.

The computer system 1900 may include a storage 1906 that holds data for indefinite periods of time. The storage 1906 may continue to hold data even when the computer system 1900 is powered down. Various types of storage 1906 are magnetic tape drives, solid state drives, and flash drives. The communication component 1908 may transmit data from the memory 1904 to and from other computer systems. For example, a communication component 1908 may connect the computer system 1900 to the internet. Alternatively, the communication component 1908 may comprise an antenna that is configured to transmit and receive data. In various embodiments, the communication component 1908 may be a Bluetooth antenna, a WIFI antenna, or the like.

The system and method described herein can capture kinematic measurements of a person while performing a movement and objects (equipment, projectile, and the like) associated with the person while performing the movement at one or more locations. The determined kinematic measurements of the person and object are then compared with one or more ideal kinematic movements of the person. Based on the ideal kinematic movement, the system and method described herein can compare the determined kinematic measurements of the person with kinematic measurements of others (professional athletes, coaches, professional/Olympic standards) who have performed the same movement. Based on the comparison, the system and method described herein recommends one or more suggestions for improvement to the person. The images of the person/object captured while performing the movement, the determined kinematic measurements of the person and object during the movement, the comparison between the person and others, and the one or more suggestions recommended are presented to the person on their respective device or a display. Thus, the person has a clear visualization of their performance, and are not dependent on a coach to continuously guide them.

Further, the system and method described herein can receive real-time feedback from the person based on their performance analyzed and presented to them. The person may provide their views, feedback, and opinions using at least one of a voice message, verbal message, email message, QR code, gesture signal, and the like. The person may also receive instant replies based on the feedback or questions asked by them. The system and method described herein may further monitor the one or more gestures (gesture signal) provided by the person based on the kinematics, comparison, goals, and suggestions presented to them. Based on the identified gesture using an AI engine, the system can prompt the person while performing the movement. For example, the prompt messages may be ‘Good Work’, ‘Keep it Up, ‘Are you okay?’, and the like. Thus, the system and method described herein may interact with the person while they are performing their movement or after their movement has been performed for determining whether they are feeling comfortable. This thus leads to an improved human-system relationship or human-machine relationship.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and such modifications are considered to be within the scope of the present disclosure.

The embodiments herein and the various features and advantageous details thereof are explained with reference to the non-limiting embodiments in the following description. Descriptions of well-known components and processing techniques are omitted so as to not obscure the embodiments herein. The examples used herein are intended merely to facilitate an understanding of ways in which the embodiments herein may be practiced and to further enable those of skill in the art to practice the embodiments herein. Accordingly, the examples may not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications may and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments, those skilled in the art will recognize that the embodiments herein can be practiced with modification within the spirit and scope of the embodiments as described herein.

Any discussion of documents, acts, materials, devices, articles or the like that has been included in this specification is solely for the purpose of providing a context for the disclosure. It is not to be taken as an admission that any of these matters form a part of the prior art base or were common general knowledge in the field relevant to the disclosure as it existed anywhere before the priority date of this application.

The numerical values mentioned for the various physical parameters, dimensions or quantities are approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the embodiments, it will be appreciated that many embodiments can be made and that many changes can be made in the embodiments without departing from the principles of the disclosure. These and other changes in the embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A system for analyzing one or more movements of moveable entity, the system comprising:
a processor in communication with a camera, wherein the processor is configured to:
determine one or more skeletal locations of the moveable entity performing a movement based on a plurality of images captured by the camera;
determine one or more kinematic measurements of the one or more skeletal locations of the moveable entity;
compare the one or more kinematic measurements of the moveable entity with an ideal kinematic movement for the moveable entity ; and
provide an analysis of the moveable entity based on the comparing of the one or more kinematic measurements of the moveable entity with the ideal kinematic movement for the moveable entity.

2. The system as claimed in claim 1, wherein the ideal kinematic movement is based on at least one of a size, a weight, an age, a gender, a limb length, a limb diameter, a limb density, a body diameter, a body density, and a skeletal makeup of the moveable entity.

3. The system as claimed in claim 1, wherein the processor is further configured to:
determine one or more location points of an object associated with the moveable entity while
performing the movement; and
determine the one or more kinematic measurements of the object based on the one or more location points.

4. The system as claimed in claim 1, wherein the processor is further configured to:
determine a first location of each skeletal location of the moveable entity;
determine one or more subsequent locations of each skeletal location while the moveable entity moves at intervals;
receive the plurality of images of the moveable entity captured by the camera at each interval; and
determine the one or more kinematic measurements at the first location and subsequent locations for a selected image at each interval.

5. The system as claimed in claim 1, further comprising a display that displays the one or more kinematic measurements during or after the moveable entity performs the movement.

6. The system as claimed in claim 1, wherein the one or more kinematic measurements are further determined based on one or more sensors that track movement of the camera.

7. The system as claimed in claim 3, wherein the processor is further configured to:
determine a first object location of the object that is associated with the moveable entity ;
determine one or more subsequent object locations of the object at intervals;
receive the plurality of images of the object captured by the camera at each interval; and
determine the one or more kinematic measurements at the first object location and subsequent object locations for a selected image at each interval.

8. The system as claimed in claim 7, further comprising a display that displays the one or more kinematic measurements of the object during or after the moveable entity is performing the movement.

9. The system as claimed in claim 1, further comprising one or more sensors that measure motion of the moveable entity; and
wherein the one or more kinematic measurements are further based on the one or more sensors.

10. The system as claimed in claim 1, wherein the processor is further configured to:
receive an input from the moveable entity based on the analysis, wherein the input comprises at
least one of a voice message, verbal message, email message, button, Bluetooth, QR code, gesture signal, or a combination thereof.

11. The system as claimed in claim 1, wherein the processor is further configured to provide a trend based on the analysis of two or more movements of the moveable entity.

12. The system as claimed in claim 11, wherein the trend comprises a rating based on the one or more kinematic measurements.

13. A method for analyzing one or more movements of a moveable entity, the method comprising:
determining, by a processor in communication with a camera, one or more skeletal locations of the moveable entity performing a movement based on a plurality of images captured by the camera;
determining, by the processor, one or more kinematic measurements of the one or more skeletal locations of the moveable entity ;
comparing, by the processor, the one or more kinematic measurements of the moveable entity with an ideal kinematic movement for the moveable entity; and
providing, by the processor, an analysis of the moveable entity based on the comparing of the one or more kinematic measurements of the moveable entity with the ideal kinematic movement for the moveable entity.

14. The method as claimed in claim 13, wherein the ideal kinematic movement is based on at least one of a size of the moveable entity, a weight of the moveable entity, an age of the moveable entity, a gender of the moveable entity, and a skeletal makeup of the moveable entity.

15. The method as claimed in claim 13, further comprising:
determining one or more location points of an object that is associated with the moveable entity
while performing the movement; and
determining the one or more kinematic measurements of the object based on the one or more location points.

16. The method as claimed in claim 15, further comprising:
determining a first location of each skeletal location of the moveable entity ;
determining one or more subsequent locations of each skeletal location while the moveable entity moves at intervals;
receiving the plurality of images of the moveable entity captured by the camera at each interval; and
determining the one or more kinematic measurements at the first location and subsequent locations for a selected image at each interval.

17. The method as claimed in claim 16, further comprising a display that displays the one or more kinematic measurements at least while the moveable entity is performing the movement or after the moveable entity has performed the movement.

18. The method as claimed in claim 15, further comprising:
determining a first object location of the object that is associated with the moveable entity;
determining one or more subsequent locations of the object at intervals;
receiving the plurality of images of the object captured by the camera at each interval; and
determining the one or more kinematic measurements at the first object location and subsequent object locations for a selected image at each interval.

19. The method as claimed in claim 18, further comprising a display that displays the one or more kinematic measurements of the object at least while the moveable entity is performing the movement or after the moveable entity has performed the movement.

20. One or more non-transitory computer-readable storage mediums storing one or more sequences of instructions, which when executed by one or more processors, causes the one or more processors to perform one or more steps of:
determining one or more skeletal locations of a moveable entity performing a movement based on a plurality of images captured by a camera in communication with the one or more processors;
determining one or more kinematic measurements of the one or more skeletal locations of the moveable entity;
determining one or more location points of an object that is associated with the moveable entity while performing the movement;
determining the one or more kinematic measurements of the object based on the one or more location points;
comparing the one or more kinematic measurements of the moveable entity and the object with an ideal kinematic movement; and
providing an analysis of the moveable entity based on the comparing of the one or more kinematic measurements of the moveable entity with the ideal kinematic movement for the moveable entity.
